(19) United States
(12) Patent Application Publication
(10) Pub. No.: US 2011/0256131 A1
(43) Pub. Date: Oct. 20, 2011

(54) BLOCKERS OF LIGHT, LTalpha1beta2 AND LTalpha2beta1 OR ITS RECEPTOR LTbetaR FOR THE PREVENTION AND TREATMENT OF CHRONIC HEPATITIS AND OTHER LIVER DISEASES

(75) Inventors: Mathias Heikenwaelder, Zuerich (CH); Adriano Aguzzi, Zuerich (CH)

(73) Assignee: UNIVERSITAET ZUERICH, Zuerich (CH)

(21) Appl. No.: 13/125,503

(22) PCT Filed: Oct. 14, 2009
(86) PCT No.: PCT/EP2009/063406

§ 371 Date: Jul. 1, 2011

(30) Foreign Application Priority Data
Oct. 22, 2008 (EP) EP 08018425.2

(51) Int. Cl. A61K 39/395; A61K 38/00; C07K 16/18; G01N 33/566; C07K 14/00; C07H 21/02; A61P 1/16; A61P 31/14; A61K 31/713; C07K 16/10

(52) U.S. Cl. 424/133.1; 424/158.1; 514/44 A; 424/172.1; 424/161.1; 514/4.3; 514/1.1; 530/389.2; 530/389.4; 530/350; 530/387.3; 536/24.5; 435/7.92

(57) ABSTRACT

The present invention relates to a method of preventing and treating chronic hepatitis and other liver diseases comprising administering a blocker of Light, LTα1β2, LTα2β1 or LTβR, and the use of such blockers in said prevention and treatment and in the manufacture of medicaments for preventing and treating chronic hepatitis and other liver diseases.

### FIELD OF THE INVENTION

This invention relates to the prevention and treatment of chronic hepatitis and other liver diseases, using blockers of Light, LTα1β2 and LTα2β1 or its receptor LTβR.

### BACKGROUND OF THE INVENTION

Lymphotoxin α (LTα), Lymphotoxin β (LTβ) and the tumor necrosis factor superfamily member 14 (TNFSF14 also called Light) are pro-inflammatory cytokines that are typically expressed by activated T-, B-, NK- and lymphoid tissue inducer cells. Whereas LTβ is constitutively expressed, LTα is inducible in T- and B-cells. LT is crucial for organogenesis and maintenance of lymphoid tissue and exists as membrane bound heterotrimers (LTα₁β₂ or LTα₂β₁) or secreted homotrimers (LTα₃). Heterotrimeric LT signals via the LTβ receptor (LTβR), whereas LTα₃ mainly triggers tumor necrosis factor receptors 1 and 2 (TNFR1; TNFR2) and the herpes virus entry mediator (HVEM). Ectopic LTαβ expression can induce lymphoid neogenesis or tissue destruction. The LTβR can also bind the pro-inflammatory cytokine Light and is therefore not exclusive for lymphotoxins. In addition Light also binds to the Herpes simplex virus entry mediator (HVEM).

This invention focuses on the prevention and treatment of chronic hepatitis and other liver diseases, especially hepatitis B virus (HBV) or hepatitis C virus (HCV) induced chronic hepatitis and hepatocellular carcinoma (HCC), liver fibrosis, liver cirrhosis, hemochromatosis, non-alcoholic steatohepatitis (NASH), chemotherapy associated hepatits (CASH), Wilson's disease, hepatosteatosis and bile duct diseases (primary sclerosing cholangitis, primary biliary cirrhosis, cholangitis). Hepatitis can be induced by autoimmune processes (autoimmune hepatitis, primary biliary cirrhosis), by alcoholic liver destruction and by infections, particularly the hepatitis viruses HAV, hepatitis B virus (HBV), hepatitis C virus (HCV), HDV, HEV, and HGV. Hepatitis turns into chronic hepatitis if the primary inflammatory stimulus cannot be resolved or does not disappear (persistent viral infection). Therefore, HCV or HBV infections frequently result in chronic hepatitis and cirrhosis, and often trigger—through largely unknown mechanisms—hepatocellular carcinoma (HCC), the most common primary liver cancer. This strong causal relationship between inflammation and liver carcinogenesis is corroborated by epidemiological and experimental data. Genetic predisposition, including polymorphisms in genes encoding LTα, IL1β, caspase-8, MMP-1, -3 and -9, cyclin D1 or the glutathione S-transferase and gender are being discussed as risk factors for HCC development.

### SUMMARY OF THE INVENTION

The present invention relates to a method of preventing and treating chronic hepatitis and other liver diseases, using blockers of the pro-inflammatory cytokines Light, LTα1β2 and LTα2β1 or its receptor LTβR. Furthermore the invention relates to blockers of the cytokines Light, LTα1β2 and LTα2β1 or its receptor LTβR for use in the prevention and treatment of chronic hepatitis and other liver diseases.

The invention further relates to a method of screening for a compound effective in the prevention and treatment of chronic hepatitis and other liver diseases, comprising contacting a candidate compound with Light, LTβR, LTα1β2 or LTα2β1 and choosing candidate compounds which selectively reduce activity of Light, LTβR, LTα1β2 or LTα2β1. The invention further relates to compounds selected by these methods of screening.

### BRIEF DESCRIPTION OF THE FIGURES

FIG. 1: Upregulation of LTβR and its ligands LTα, LTβ and TNFSF14/LIGHT in HBV or HCV infected human livers and in HCC.

(a) Real-time PCR analysis for Itα, Itβ, tnfsf14/light and Itβr mRNA expression in human livers derived from healthy liver controls, hepatitis B or C biopsies and HCC. Increase in Itα, Itβ and Itβr mRNA was 27 to 29 fold in average in hepatitis B and C infected livers or HCC when compared to controls (p<0.001 for Itα, Itβ and Itβr). Tnfsf14/light upregulation was ca. 23 and 25 fold (p<0.001) in hepatitis B and C infected livers or HCC when compared to controls.

(b) Box blot analysis depicting age and gender distribution. y=age in years.

FIG. 2: Characterization of tg1223 livers at 3 and 9 months of age.

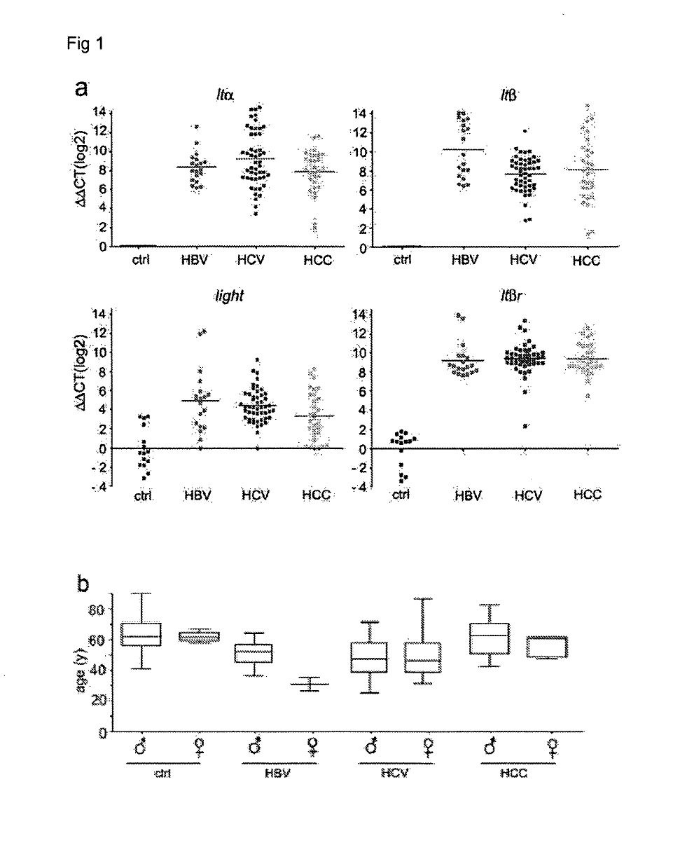

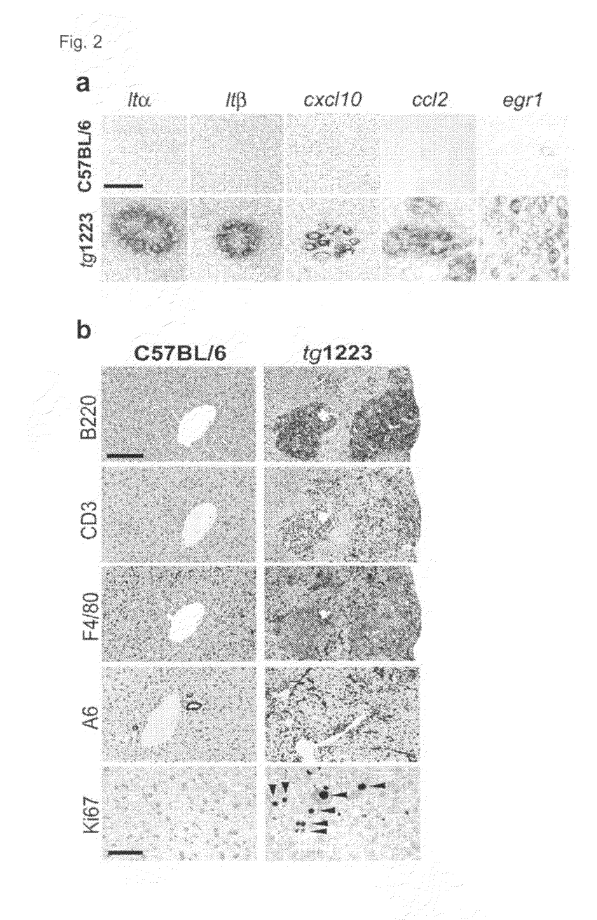

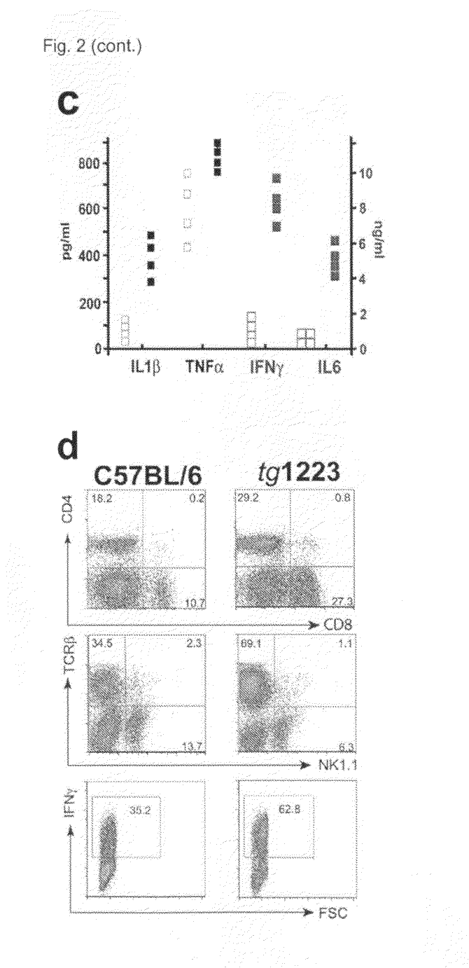

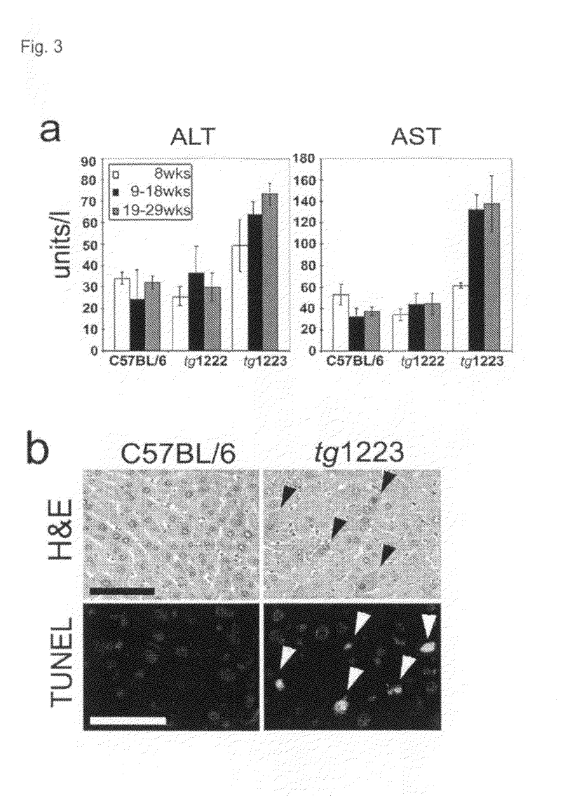

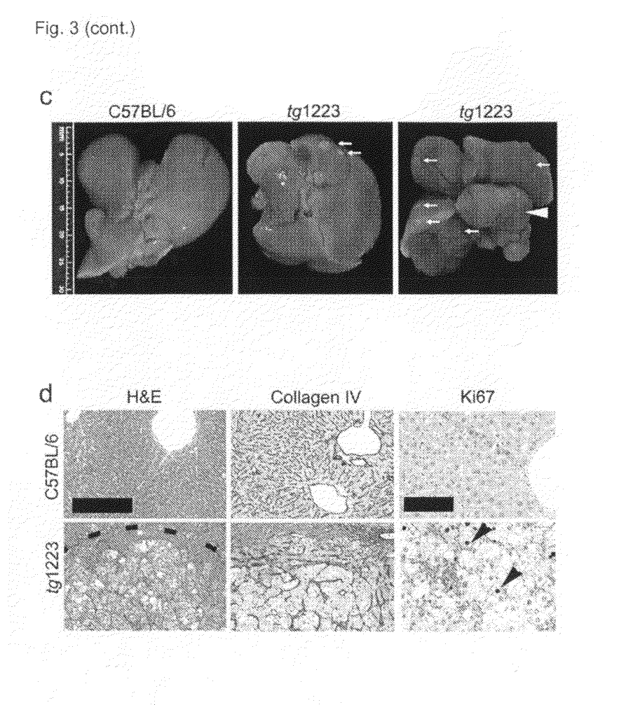

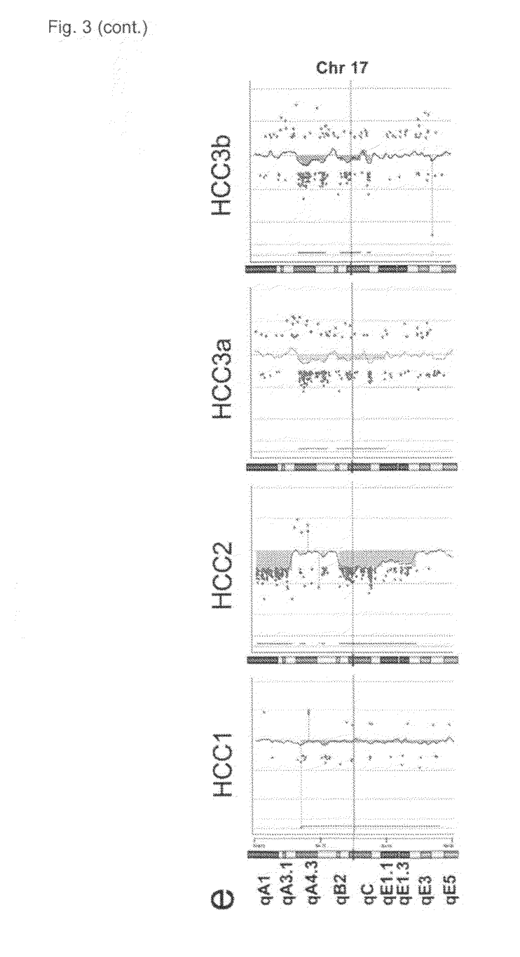

(a) In situ hybridization of liver cryosections from C57BL/6 and tg1223 mice for Itα, Itβ, cxcl10, ccl2 and egr1 mRNA expression (3 months). Multiple scattered foci of hepatocyte specific Itα, Itβ, cxcl10, ccl2 and egr1 mRNA expression were detected (scale bar: 50 μm).

(b) Immunohistochemical analysis of representative paraffin sections of 9 months old C57BL/6 and tg1223 livers. 8220 for B-cells, CD3 for T-cells, F4/80 for macrophages and Kupffer cells, A6 for oval cells. In contrast to C57BL/6, tg1223 livers displayed portal and lobular inflammatory infiltrates with B- and T-cells and activated Kupffer cells. Oval cell proliferation was observed in tg1223 livers (scale bar: 150 μm). Numerous Ki67+proliferating hepatocytes (arrow heads) and inflammatory cells were detected in tg1223 livers (scale bar: 50 μm).

(c) ELISA for IL1β, TNFα, IFNγ and IL6 in C57BL/6 (hollow symbols) and tg1223 (filled symbols) liver homogenates (9 months). Elevated levels of IL1β (p<0.05), IFNγ (p<0.05), IL6 (p<0.05) were detected in tg1223 livers. Differences for TNFα were less dramatic.

(d) Intrahepatic CD8 positive, CD4 positive, TCRβ positive, 107 positive and NK1.1 positive lymphocytes were analyzed by flow cytometry at 9 months of age. CD4 positive, CD8 positive and TCRβ positive T-cells were increased in tg1223 livers, whereas NK1.1 positive or IL17 positive cells were slightly reduced or remained unchanged. Numbers in each quadrant indicate the relative percentage of cells analyzed. Staining intensity is depicted in a log scale. FSC: Forward scatter.

FIG. 3: Chronic liver injury and HCC development in tg1223 mice.

(a) From the age of 9 weeks (wks) on, aminotransferases were significantly elevated (p<0.03) in sera of mice overexpressing Lymphotoxin alpha and beta in the liver (AST>ALT).

(b) Increased hepatocyte cell death was observed in tg1223 livers by H&E and TUNEL/DAPI staining at around 9 months of age. Black arrowheads indicate apoptotic hepatocytes. White/TUNEL+hepatocyte nuclei demonstrate apoptosis (white arrowheads) (scale bars: 50 μm).

(c) Macroscopic analysis of 18 months old C57BL/6 (left panel) and tg1223 livers at the age of 12 (middle panel) and 18 months (right panel). No macroscopically detectable tumors were found in any of the investigated C57BL/6 livers. Tumors ranged from small (1-25 mm) nodules (middle panel; white arrows) to affecting whole liver lobes (right panel, white arrow head).

(d) Histological analysis of C57BL/6 livers and HCC found in tg1223 livers (12 months) (H&E, collagen IV and Ki67 staining). H&E stained liver sections with dashed lines depicting tumor borders. Collagen IV staining highlights the broadening of the liver cell cords indicative of HCC in tg1223 livers. In comparison, C57BL/6 mice showed intact liver cell cords (i.e. one to two cells wide) (scale bar: 200 μm, left and middle column). Abnormal Ki67+proliferating hepatocytes were only found in tg1223 livers (arrowheads; scale bar: 100 μm, right column).

(e) Chromosomal aberrations in 4 individual tg1223 HCC were detected by array genomic hybridization analysis (aCGH). 2 among those HCC originated in different lobes of the same liver (HCC 3a and 3b). The q-arm of chromosome 17 served as an example for localized chromosomal aberrations. Shown are the log ratios of C57BL/6 signal versus tg1223 signal intensities. Negative log ratios represent gains of genetic material in a given HCC whereas positive log ratios correspond to losses. Only data points are shown that exceed an absolute signal intensity of a log ratio of 0.5, which was the background level of C57BL/6 controls. The lines represent smoothed moving averages taking into account all log ratios. Estimated copy number aberrations are indicated with shaded surfaces.

### DETAILED DESCRIPTION OF THE INVENTION

The present invention relates to a method of preventing and treating chronic hepatitis and other liver diseases, comprising administering blockers of Light, LTα1β2 and LTα2β1 or its receptor LTβR, and the use of such blockers in said prevention and treatment and in the manufacture of medicaments for preventing and treating chronic hepatitis and other liver diseases.

The action of Light, LTα1β2 and LTα2β1 can be blocked by administration of LTβR-Fc, of antibodies or antibody fragments directed against LTα1β2, LTα2β1 or Light, of molecules that affect the protein or mRNA expression of LTβR, LTα1β2, LTα2β1 or Light (siRNA; miRNA), as well as of small molecules that interfere with the binding of ligands to LTβR, and of Light to HVEM. The production of LTβR, LTα1β2, LTα2β1 or Light can be inhibited by using siRNA in vitro but also by directly suppressing the promoter activity of LTβR, LTα1β2, LTα2β1 or Light with small molecules or suppressors of the transcription factors involved in LTβR, LTα1β2, LTα2β1 or Light transcription regulation. The action of LTα1β2, LTα2β1 and Light can be inhibited by LTβ receptor blockers. Additionally, targeting of the LTβR pathway can be achieved by the administration of neutralizing antibodies or antibody fragments to Light, LTα1β2, LTα2β1 or the LTβ receptor or by proteins, protein analogs or small synthetic compounds which bind LTα1β2, LTα2β1 and/or Light, and thereby prevent its binding to the LTβ receptor, or bind to the LTβ receptor. A further way to prevent binding to the LTβ receptor is to use soluble LTβ receptor or fragments thereof. A way to prevent binding of Light to HVEM is to use soluble HVEM or fragments thereof.

Examples of Light, LTα1β2, LTα2β1 and LTβR blockers according to the invention are disclosed in the following. However, the invention is not restricted to the blockers disclosed therein, but extends to all blockers of Light, LTα1β2, LTα2β1 and LTβR or molecules that interfere with the expression levels or the activity of Light, LTα1β2, LTα2β1 and LTβR.

Preferred blockers of Light, LTα1β2, LTα2β1 and LTβR according to the invention are:
Soluble LTβR (for Light, LTα and LTβ) or soluble HVEM (for Light); for example LTβR-Fc, a fusion protein consisting of the human LTβR fused to the constant human immunoglobulin portion Fc (baminercept-alfa, Biogen Inc, Cambridge Mass., USA) as well as the compounds described in U.S. Pat. No. 7,255,854.
Antibodies that bind to LTα or LTβ alone, LTα3, LTα1β2 and/or LTα2β1 or Light, antigen binding fragments of an antibody (e.g. Fab fragments) or antibody-like molecules (e.g. repeat proteins) which by binding to LTα or LTβ alone, Light, LTα1β2 and/or LTα2β1 deplete LTα1β2 and/or LTα2β1 and/or Light from the extracellular space or that block the binding between LTβR and its ligands. Antibodies against recombinant human LTα are state of the art and include the well characterized antibodies 9B9, NC2, AG9, FF2, AA6, GC4, AH6, DH1, CH12, FE2, BF7, and BMSIO5 (Browning J L, Dougas I, Ngam-ek A, Bourdon P R, Ehrenfels B N, Miatkowski K, Zafari M, Yampaglia A M, Lawton P, Meier W, et al., J. Immunol. 1995, 154:33-46). Antibodies against recombinant human LTβ include the well characterized antibodies B9, 827, c37, and A3. (Browning J L et al., loc. cit.). These antibodies are available from Biogen.
Antibodies, antigen binding fragments of an antibody (e.g. Fab fragments) or antibody-like molecules (e.g. repeat proteins) which by binding to LTβR block the action of LTα1β2, LTα2β1 and/or Light. Such antibodies preferably bind to LTβR in the region where LTα1β2, LTα2β1 or Light would normally bind, but without inducing LTβR-signaling. Such antibodies include LLTB1 (Anand Set al., J. Clin. Invest. 116 (4): 1045-1051 (2006).
Virus-like particles loaded with LTβR, Light LTα or LTβ and therefore inducing an antibody response directed against these molecules with the effect to block their biological activity.
Antisense molecules for downregulation of LTβR, Light, LTα or LTβ. These antisense molecules are 12-50 nucleotides in length and encode a given sequence found in the exons or introns of Light, LTα, LTβ, or LTβR. Moreover, antisense molecules containing a sequence of the Light, LTα, LTβ, or LTβR promoters and binding within the promoter region may be used. Finally, antisense molecules binding in the 3′ UTR-non translated regions of Light, LTα, LTβ, or LTβR are contemplated. Compounds that modulate the expression of Light are e.g. described in US 2004/0096835.
Small molecules that inhibit LTα1β2, LTα2β1 or the binding of Light to LTβR. Small molecules contemplated are synthetic compounds up to a molecular weight of 1000 which have suitable physiological activity and pharmacological properties making them useful for the application as medicaments. Such small synthetic molecules are, for example, found by the screening method of the present invention described below. Alternatively, such small molecules are designed by molecular modelling taking into account possible binding sites of LTα, LTβ, Light and LTβR.
Proteins and protein analogs which bind LTα1β2, LTα2β1 and/or Light and thereby prevent its binding to the LTβ receptor, or bind to the LTβ receptor are, for example, synthetic proteins or protein analogs which mimic the variable region scFv of binding and/or neutralizing antibodies, or antibodies that mimic a binding pocket for LTα or LTβ of the LTβR. Likewise small molecules could be applied, which mimic the variable region scFv of binding and/or neutralizing antibodies, or that mimic a binding pocket for LTα or LTβ of the LTβR.

Most preferred blocker is baminercept-alfa.

One aspect of the invention relates to a method of preventing and treating chronic hepatitis and other liver diseases, comprising administering blockers of Light, LT-a1β2, LTα2β1 or LTβR as defined hereinbefore in a quantity effective against chronic hepatitis and other liver diseases to a mammal in need thereof, for example to a human requiring such treatment. The treatment may be for prophylactic or therapeutic purposes. For the administration, the blocker is preferably in the form of a pharmaceutical preparation comprising the blocker in chemically pure form and optionally a pharmaceutically acceptable carrier and optionally adjuvants. The blocker is used in an amount effective against chronic hepatitis and other liver diseases. The dosage of the active ingredient depends upon the species, its age, weight, and individual condition, the individual pharmacokinetic data, the mode of administration, and whether the administration is for prophylactic or therapeutic purposes. In the case of an individual having a bodyweight of about 70 kg the daily dose administered is from approximately with 0.1 mg/kg to approximately 1000 mg, preferably from approximately 0.5 mg to approximately 100 mg/kg, of a blocker of Light, LTα1β2, LTα2β1 or LTβR. An example is published in Gommerman J L, Mackay F, Donskoy E, Meier W, Martin P, and Browning J L, J. Clin. Invest. 2002, 110(9):1359-69.

Pharmaceutical compositions for enteral administration, such as nasal, buccal, rectal or, especially, oral administration, and for parenteral administration, such as subcutaneous, intravenous, intrahepatic or intramuscular administration, are especially preferred. The pharmaceutical compositions comprise from approximately 1% to approximately 95% active ingredient, preferably from approximately 20% to approximately 90% active ingredient.

For parenteral administration preference is given to the use of solutions of the blockers of Light, LTα1β2, LTα2β1 or LTβR, and also suspensions or dispersions, especially isotonic aqueous solutions, dispersions or suspensions which, for example, can be made up shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

For oral pharmaceutical preparations suitable carriers are especially fillers, such as sugars, for example lactose, saccharose, mannitol or sorbitol, cellulose preparations and/or calcium phosphates, and also binders, such as starches, cellulose derivatives and/or polyvinylpyrrolidone, and/or, if desired, disintegrators, flow conditioners and lubricants, for example stearic acid or salts thereof and/or polyethylene glycol. Tablet cores can be provided with suitable, optionally enteric, coatings. Dyes or pigments may be added to the tablets or tablet coatings, for example for identification purposes or to indicate different doses of active ingredient. Pharmaceutical compositions for oral administration also include hard capsules consisting of gelatin, and also soft, sealed capsules consisting of gelatin and a plasticizer, such as glycerol or sorbitol. The capsules may contain the active ingredient in the form of granules, or dissolved or suspended in suitable liquid excipients, such as in oils.

Transdermal/intraperitoneal and intravenous applications are also considered, for example using a transdermal patch, which allows administration over an extended period of time, e.g. from one to twenty days.

Intravenous or subcutaneous application are particularly preferred.

Another aspect of the invention relates to the use blockers of Light, LTα1β2, LTα2β1 or LTβR as described hereinbefore in the prevention and treatment of chronic hepatitis and other liver diseases, and in the manufacture of medicaments for treating these diseases.

“Chronic hepatitis” is especially hepatitis B virus (HBV) or hepatitis C virus (HCV) induced chronic hepatitis.

“Other liver diseases” are, for example, hepatocellular carcinoma (HCC), liver fibrosis, liver cirrhosis, hemochromatosis, non-alcoholic steatohepatitis (NASH), chemotherapy associated hepatits (CASH), Wilson's disease, hepatosteatosis and bile duct diseases, such as primary sclerosing cholangitis, primary biliary cirrhosis, and cholangitis.

Medicaments according to the invention are manufactured by methods known in the art, especially by conventional mixing, coating, granulating, dissolving or lyophilizing.

The blockers of Light, LTα1β2, LTα2β1 or LTβR can be administered alone or in combination with one or more other therapeutic agents, possible combination therapy taking the form of fixed combinations of a blocker of Light, LTα1β2, LTα2β1 or LTβR and one or more other therapeutic agents known in the prevention or treatment of chronic hepatitis and other liver diseases, the administration being staggered or given independently of one another, or being in the form of a fixed combination.

Possible combination partners considered are Interferon, and Lamivudine and other nucleoside or nucleotide analogues.

The invention further relates to a method of screening for a compound effective in the prevention and treatment of chronic hepatitis and other liver diseases comprising contacting a candidate compound with Light, LTα1β2, LTα2β1 or LTβR and choosing candidate compounds which selectively reduce the activity of Light, LTα1β2, LTα2β1 or LTβR. The invention further relates to compounds selected by these methods of screening.

Blockers of Light, LTα1β2, LTα2β1 or LTβR activity are identified by contacting Light, LTα1β2, LTα2β1 or LTβR with a candidate compound. A control assay with the corresponding Light, LTα1β2, LTα2β1 or LTβR in the absence of the candidate compound is run in parallel. A decrease in activity in the presence of the candidate compound compared to the level in the absence of the compound indicates that the candidate compound is a Light, LTα1β2, LTα2β1 or LTβR blocker.

Antibodies against Light, LTα1β2, LTα2β1 or LTβR can be generated e.g. by immunization of LT or Light knockout mice by using the virus like particle system, or by injection of recombinant protein in knockout mice.

### Concepts and Evidence Behind the Invention

Hepatitis B and C viruses are the major cause of chronic hepatitis and hepatocellular carcinoma (HCC), the most common primary liver cancer in humans. By real-time PCR and also by ELISA, drastically overexpressed lymphotoxin (LT) α and β, TNFSF14/LIGHT and lymphotoxin-β receptor (LTβR) was found in liver biopsies of chronic hepatitis B or C and in HCC. Overexpression of LTα and β in mouse livers caused pathologies similar to viral hepatitis in humans. After ≧12 months, about 30% of LT overexpressing mice developed HCC, which was prevented by lymphocyte ablation. Similarly, removal of IKKβ from hepatocytes, but not depletion of TNFR1, prevented both hepatitis and HCC development. Acute in vivo LTβR stimulation identified hepatocytes as the major responsive liver cell type and mirrored in part transcriptional changes of LT over-expressing livers. The data indicate that deregulated LTβR signaling on hepatocytes is involved in the pathogenesis of chronic hepatitis and HCC. Therefore, interference with Light, LTα, LTβ as well as with LTβR or IKKβ signaling is of therapeutic value in these conditions.

### Experiments Performed

Upregulation of Itβr, Itα, and tnfsf14/Light mRNA in HBV or HCV Affected Livers and in HCC

Transcriptional levels of the pro-inflammatory cytokines LTα, LTβ, Light and LTβR were measured in human HBV (n=19) or HCV-infected (n=49) livers as well as in HCC (n=30) and compared to healthy controls (n=15). Itα, Itβ, light and Itβr transcripts were significantly (P<0.001) elevated in HBV or HCV infected livers and in HCC. Expression levels were independent of gender (P<0.001), age (P<0.001) and HCV genotypes (1, 2 or 3).

These results indicate a possible involvement of enhanced and persistent hepatic LTβR signaling in chronic hepatitis and, potentially, in hepatocellular carcinogenesis. This was addressed by generating transgenic mice that overexpress LTα and β specifically on hepatocytes at low (tg1222) and high levels (tg1223). These mice had severe chronic hepatitis at ≧9 months of age leading to a ˜35% prevalence of HCC, similar to human viral hepatitis.

To elucidate the mechanisms inducing chronic hepatitis and HCC formation, tg1223 mice were intercrossed with tnfr1^{-/-}, IKKβ^{Δhep} or rag1^{-/-} mice. Surprisingly it was found that both the LTαβ-induced chemokine and cytokine storm, the development of chronic hepatitis, and even HCC formation were completely abolished by the ablation of lymphocytes or of hepatocyte-specific IKKβ. Hence, rather than directly acting as a cell-autonomous oncogene on hepatocytes, LTαβ exerts its carcinogenic effect through the recruitment of lymphocytes.

The signaling mechanisms driving liver inflammation and cancer development in tnfrf1^{-/-} mice and preventing these conditions in IKKβ^{Δhep} mice were further investigated. Acute i.v. administration of the LTβR agonist 3C8 into C57BL/6or tnfr1^{-/-} mice induced hepatic changes similar to those seen in tg1223 mice at 3 months. This indicated that LTβR signaling induces p65 translocation and upregulation of selected NF-κB target genes even in the absence of TNFR1. Therefore, heterotrimeric LTαβ suffices to induce chronic inflammation and liver cancer through a pathway that is independent of TNFR1. In contrast i.v. administration of 3C8 to IKKβ^{Δhep} mice did neither lead to hepatic p65 translocation nor to the upregulation of selected target genes. This explains why tg1223/IKKβ^{Δhep} mice fail to upregulate liver specific cytokines and chemokines and why they do not develop chronic hepatitis and liver cancer. Importantly, these experiments identified hepatocytes as the major entity for integrating LTβR signaling in the liver.

### Example of Treatment

The following groups of patients are treated with blockers of LTα, LTβ, LTβR, or Light:
1) Patients diagnosed to be serum positive for HBV or HCV but that do not exhibit chronic hepatitis.
2) Patients diagnosed to be serum positive for HBV or HCV that exhibit chronic hepatitis.
3) Patients diagnosed to be serum positive for HBV or HCV and have cirrhosis.
4) Patients diagnosed to be serum positive for HBV or HCV and that suffer from HCC on the basis of cirrhosis.
5) Patients diagnosed to be serum positive for HBV or HCV and that suffer from HCC without cirrhosis.

Treatment is performed on a weekly basis intravenously or subcutaneously, preferably with baminercept-alfa (40 mg/kg on average, more or less dependent on the stage of disease). Other GLP based products as defined above and blocking Light, LTα1β2 and/or LTα2β1 or its receptor LTβR may be applied to the patients for the prevention and treatment of chronic hepatitis and hepatocellular carcinoma. Patients are followed up by analyzing ALT, AST serum levels, alpha-fetoprotein serum levels, the quality of life of the treated patients, MRI scans for liver, sonography of livers and the tumor marker gp73 in serum.

1. A blocker of the cytokines Light, LTα1β2 and LTα2β1 or its receptor LTβR for use in the prevention and treatment of chronic hepatitis and other liver diseases.

2. A blocker according to claim 1 selected from LTβR-Fc, antibodies and antibody fragments directed against LTα1β2, LTα2β1 or Light, molecules that affect the protein or mRNA expression of LTβR, LTα1β2, LTα2β1 or Light, and small molecules that interfere with the binding of LTβR, LTα1β2, LTα2β1 or Light to LTβR, or of Light to HVEM.

3. A blocker according to claim 1 which is siRNA inhibiting the production of LTβR, LTα1β2, LTα2β1 or Light.

4. A blocker according to claim 1 which is a suppressor of the transcription factors involved in LTβR, LTα1β2, LTα2β1 or Light transcription regulation.

5. A blocker according to claim 1 which is a neutralizing antibody or antibody fragment to Light, LTα1β2, LTα2β1 or the LTβ receptor.

6. A blocker according to claim 1 which is a protein, protein analog or small synthetic compounds which binds to LTα1β2 , LTα2β1, or Light and thereby prevents its binding to the LTβ receptor, or binds to the LTβ receptor.

7. A blocker according to claim 1 which is a soluble LTβ receptor or a fragment thereof, or soluble HVEM or a fragment thereof.

8. A blocker according to claim 1 which is a fusion protein consisting of the human LTβR fused to the constant human immunoglobulin portion Fc.

9. A blocker according to claim 1 which is an antibody selected from the group consisting of 9B9, NC2, AG9, FF2, AA6, GC4, AH6, DH1, CH12, FE2, BF7, BMSIO5, B9, 827, c37, and A3.

10. A method of claim 15, wherein the liver diseases are hepatocellular carcinoma (HCC), liver fibrosis, liver cirrhosis, hemochromatosis, non-alcoholic steatohepatitis (NASH), chemotherapy associated hepatits (CASH), Wilson's disease, hepatosteatosis and bile duct diseases.

11. A method of claim 15, wherein hepatitis B and C are treated and prevented.

12. A method of claim 15, wherein the liver disease is hepatocellular carcinoma.

13. A method of screening for a compound effective in the prevention and treatment of chronic hepatitis and other liver diseases, comprising contacting a candidate compound with Light, LTβR, LTα1β2 or LTα2β1 and choosing candidate compounds which selectively reduce activity of Light, LTβR, LTα1β2 or LTα2β1.

14. A compound selected according to the method of claim 13.

15. A method of preventing and treating chronic hepatitis and other liver diseases, comprising administering blockers of Light, LTα1β2 and LTα2β1 or its receptor LTβR in a quantity effective against chronic hepatitis and other liver diseases to a mammal in need thereof.

16. A method of claim 15, wherein the blockers are selected from LTβR-Fc, antibodies and antibody fragments directed against LTα1β2, LTα2β1 or Light, molecules that affect the protein or mRNA expression of LTβR, LTα1β2, LTα2β1 or Light, and small molecules that interfere with the binding of LTβR, LTα1β2, LTα2β1 or Light to LTβR, or of Light to HVEM.

17. A blocker according to claim 2 which is siRNA inhibiting the production of LTβR, LTα1β2, LTα2β1 or Light.

18. A blocker according to claim 2 which is a suppressor of the transcription factors involved in LTβR, LTα1β2, LTα2β1 or Light transcription regulation.

19. A blocker according to claim 2 which is a neutralizing antibody or antibody fragment to Light, LTα1β2, LTα2β1 or the LTβ receptor.

20. A blocker according to claim 2 which is a protein, protein analog or small synthetic compounds which binds to LTα1β2 , LTα2β1, or Light and thereby prevents its binding to the LTβ receptor, or binds to the LTβ receptor.
